(19) United States
(12) Patent Application Publication
(10) Pub. No.: US 2010/0286527 A1
(43) Pub. Date: Nov. 11, 2010

(54) ULTRASOUND SYSTEM WITH MULTI-HEAD WIRELESS PROBE

(75) Inventors: Michael G. Cannon, Haverford, PA (US); Joseph A. Urbano, Audubon, PA (US); Kevin S. Randall, Ambler, PA (US); Lawrence A. Engle, Stowe, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Penrith Corporation, Plymouth Meeting, PA (US)

(21) Appl. No.: 12/437,990

(22) Filed: May 8, 2009

(51) Int. Cl. A61B 8/14

(52) U.S. Cl. 600/459

(57) ABSTRACT

An ultrasound system and method is described in which a probe is used in conjunction with a main unit. The probe contains at least two different transducer or transducer array types to provide an operator with a selection of transducer types for use without having to change to a different probe. Transducer types may include wide-band and narrow band transducers or transducer arrays within a single probe, and may be selected for use by a selector switch on the probe. Data collected by the probe during operation may be transmitted wirelessly back to a main unit through the use of a wireless antenna incorporated into the probe. In addition, one of the transducers at either end of the probe may be replaced by an adjunct equipment type such as a stethoscope.

### BACKGROUND

Ultrasound users often require several different transducers to cover a variety of imaging applications. These transducers vary in several parameters such as bandwidth, center frequency, array dimensions, and curvature. Higher center frequency provides improved lateral resolution at the expense of depth of penetration. The axial resolution is improved through higher bandwidths. Larger arrays provide improved focus at deeper depths but may not be appropriate for areas of the body with limited soft-tissue access such as through the rib cage. Array curvature provides a wider field of view in the far-field.

Current ultrasound imaging machines are supplied with imaging transducers of high fractional bandwidth to allow then to cover a large range of center frequencies. Fractional bandwidth is the ratio of signal bandwidths to center frequency. Typical fractional bandwidths are 60% to 70% (at −6 dB). In order to improve lateral resolution, conventional ultrasound machines use the broad transducer bandwidth to support an imaging mode where the system drives the transducers in such a way that the emitted spectrum is pushed to the upper edge of the transducer's frequency range. Received echo signals are filtered to this same frequency range. Because this raises the center frequency of the resulting echo spectrum, it improves lateral resolution, although it does so at the expense of the signal's bandwidth, which somewhat compromises axial resolution. In addition, because body attenuation is greater at higher frequencies and the transducer is less efficient at the band edge, it also sacrifices sensitivity (SNR) which limits penetration.

Also supported by conventional ultrasound machines is the ability to force the transducer to operate at the lower edge of its frequency range. This can improve sensitivity due to the lower attenuation at lower frequencies, although at the expense of both axial and lateral resolution.

### SUMMARY

The disclosed embodiments include a method, system, and device for conducting ultrasound interrogation of a medium using two or more different transducers in one probe housing. The novel method includes transmitting a non-beamformed or beamformed ultrasound wave into the medium using a single probe housing that provides a user the ability to quickly change the desired field focus and view without having to change probe assemblies. The novel method may further transmit the multi-configured digital data in a single data stream. In some embodiments, the transmitting may be wireless. The novel device may include a range of transducer elements to cover various imaging applications. Two or more different transducers may be provided within a single probe housing to span a broader range of applications and investigations with one probe. In an exemplary embodiment, a wireless transducer may be incorporated within the single probe, eliminating the need for a cable attached to the probe housing. In some embodiments various transducer types may be supported within the single probe housing including, but not limited to, linear arrays, phased arrays, and curvilinear arrays. In addition, in certain embodiments different frequency ranges may be offered on the same probe assembly with either the same or different transducer types.

In other embodiments, adjunct uses for the one probe may include providing a stethoscope in the same probe housing as a transducer. The acoustic signal of the stethoscope is picked up by a microphone in the transducer housing where it is amplified, digitized, and transmitted wirelessly. The wireless signal is transmitted over the same wireless link used for ultrasound signal information, and both signals are packetized, interleaved and transmitted to the main ultrasound system in a single data stream.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a block diagram illustrating various components of each transducer within an example probe;

FIG. 2 is a block diagram illustrating various components of a main unit;

FIG. 3 is a block diagram of a system for transmitting an acoustic transmit focused wave;

FIG. 4 is a block diagram of a system for receiving an acoustic transmit focused wave;

FIGS. 5A-5C provide an example of different possible configurations and techniques for providing interrogation of a medium;

FIGS. 6A-6B provide an example of different possible configurations of probe transducers within a single probe;

FIG. 7 is an example of generating and transmitting interleaved signals from an example configuration of transducers within a single probe;

FIG. 8 is an example of a combination of a microphone and a transducer within a single probe;

FIG. 9 is a diagram of a data interleaving technique;

FIG. 10 is a flow diagram of a method for establishing a link between a probe and a main unit;

FIG. 11 is a flow diagram of an inactivity timeout;

FIG. 12 is a block diagram illustrating data merger and adaptive control.

### DETAILED DESCRIPTION

The subject matter of the described embodiments is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Similarly, with respect to the components shown in the Figures, it should be appreciated that many other components may be included with the scope of the embodiments. The components are selected to facilitate explanation and understanding of the embodiments, and not to limit the embodiments to the components shown.

There are many transducer array systems contemplated by the disclosed embodiments. Most of the description focuses on a description of a diagnostic medical ultrasound system, however the disclosed embodiments are not so limited. The description focuses on diagnostic medical ultrasound systems solely for the purposes of clarity and brevity. It should be appreciated that disclosed embodiments apply to numerous other types of methods and systems.

In a transducer array system, the transducer array is used to convert a signal from one format to another format. For example, with ultrasound imaging the transducer converts an ultrasonic wave into an electrical signal, while a RADAR system converts an electromagnetic wave into an electrical signal. While the disclosed embodiments are described with reference to an ultrasound system, it should be appreciated that the embodiments contemplate application to many other systems. Such systems include, without limitation, RADAR systems, optical systems, audible sound reception systems. For example, in some embodiments, the audible sound reception system may be used at a sporting event to detect on-field sounds with a large microphone and wirelessly transmit the sound back to a main unit.

In addition, although the disclosed embodiments are described with reference to a medical ultrasound system, it should be appreciated that the embodiments contemplate application to many other types of ultrasound system. For example, the disclosed embodiments apply to non-destructive testing systems. Such non-destructive testing systems may be used to inspect metal, wood, plastics, etc. for structural integrity and/or to ascertain certain characteristics of the material. For example, the embodiments may be used to inspect piping for cracks and/or to determine their thickness. Also, non-destructive testing systems may be used to inspect material connections, like metal welds, and the like.

Also, although the disclosed embodiments are described with reference to a diagnostic system, it should be appreciated that the embodiments contemplate application to many other types of systems, including, for example, therapeutic ultrasound systems.

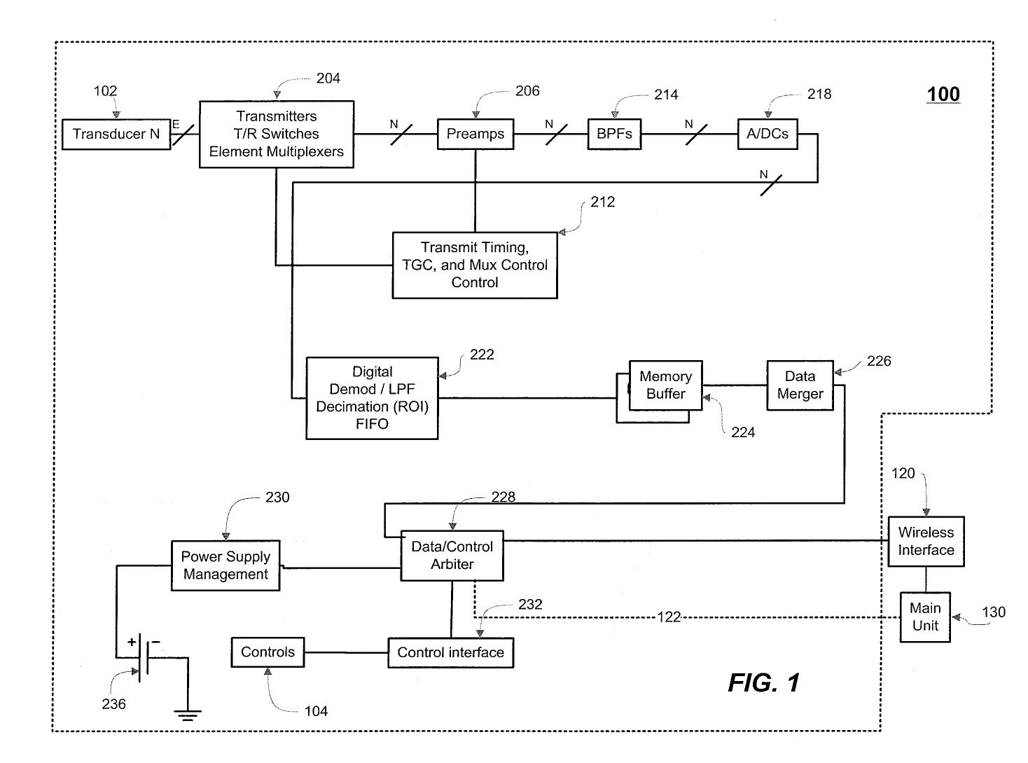

FIG. 1 is a block diagram illustrating various components of an example probe 100 according to one embodiment. It should be appreciated that any or all of the components illustrated in FIG. 1 may be disposed within a housing (not shown in FIG. 1) having any form factor. Probe 100 may include circuitry that is represented in FIG. 1 as a series of blocks, each having a different function with respect to the operation of probe 100. While the following discussion treats each of the blocks as a separate entity, an embodiment contemplates that any or all of such functions may be implemented by hardware and/or software that may be combined or divided into any number of components. For example, in one embodiment the functions represented by any or all of the blocks illustrated in FIG. 1 may be performed by components of a single printed circuit board or the like.

Transducer 102 represents any number of transducer elements that may be present in probe 100. Electroacoustic ultrasound transducer types include piezoelectric, piezoceramic, capacitive, microfabricated, capacitive microfabricated, piezoelectric microfabricated, and the like. Some embodiments may include transducers for sonar, radar, optical, audible, or the like. Transducer 102 elements may be comprised of individual transmitter and receiver elements. For example, transmitter 204 includes one or more transmitters that drive each of the transducer elements represented by transducer 102, as well as transmit and/or receive switch circuitry that isolates transmitter 204 from a receiver channel (which may be part of preamp 206 in FIG. 1) during the transmit event. The transmitters may produce a focused, unfocused or defocused transmit beam, depending on the intended application. For example, the focused beam may be useful when high peak acoustic pressure is desired as is the case of harmonic imaging. One embodiment uses defocused transmit beams to provide insonfication or interrogation of a relatively larger spatial region as required for synthetic transmit focusing. The transmit beam may be configured to elicit return echo information that is sufficient to produce an ultrasound image along an imaging plane.

Probe 100 receiver circuitry (not shown in FIG. 1) may include a low-noise, high-gain preamplifier 206 for each receive channel (e.g., manufactured by Texas Instruments model number VCA2615 dual-channel variable gain amplifier or the like). Any number of receive channels may be present in an embodiment. Preamplifier 206 may provide variable gain throughout a data acquisition time interval. Preamplifier 206 may be followed by bandpass filter 214 that may operate to reduce the noise bandwidth prior to analog-to-digital (A/D) conversion.

Transmit timing, time-gain control (TGC) and multiplexer control 212 may in some embodiments provide timing and control of each transmit excitation pulse, element multiplexer setting, and TGC waveform. An example unipolar transmitter channel circuit may include, for example, a transistor functioning as a high-voltage switch followed by a capacitor. The capacitor may be charged to a high voltage (e.g., 100V), and then discharged through the transistor upon excitation by a trigger pulse. Similar transistor-based switches may also be used for transmit/receive isolation, element-to-channel multiplexing, etc. Other embodiments may include more sophisticated transmitters capable of bipolar excitations and/or complex wave shaping and/or the like.

To focus the transmitted ultrasound energy at a desired spatial location, in some embodiments, the excitation pulse of each transducer element may be delayed in time relative to the other elements. Such a delay pattern may cause the ultrasound waves from excited elements to combine coherently at a particular point in space, for example. This may be beneficial for a focused and/or an acoustic transmit focused system, for example. Alternatively, the transmit waveforms may be delayed in such a way as to defocus the beam. This may be beneficial for a system employing synthetic transmit focusing, for example.

In some embodiments, a TGC portion of block 212 may provide a programmable analog waveform to adjust the gain of variable gain preamplifier 206. The analog waveform may be controlled by a user through a user interface such as, for example, a set of slide controls used to create a piece-wise linear function. In some embodiments, this piece-wise linear function may be calculated in software, and then programmed into sequential addresses of a digital memory, for example. The digital memory may be read out sequentially at a known time interval beginning shortly after the transmit excitation pulse, for example. In some embodiments, output of the memory may be fed into a digital-to-analog converter (DAC) to generate the analog waveform. In some embodiments, time may be proportional to the depth of the ultrasound echoes in the ultrasound receiver. As a result, echoes emanating from tissue deep within a patient's body may be attenuated more than those from shallow tissue and, therefore, require increased gain. The controlling waveform may also be determined automatically by the system by extracting gain information from the image data, for example. Also, in some embodiments, the controlling waveform may be predetermined and stored in the memory, and/or determined during system operation.

One embodiment may include a multiplexer within block 204 for multiplexing a relatively large array of transducer 102 elements into a smaller number of transmit and/or receive channels. Such multiplexing may allow a smaller ultrasound aperture to slide across a full array on successive transmit events. Both transmit and receive apertures may be reduced to the same number of channels or they may differ from each other. For example, the full array may be used for transmitting while a reduced aperture may be used on receive. It should be appreciated that any combination of full and/or decimated arrays on both transmit and receive are contemplated by the disclosed embodiments.

Multiplexing also may provide for building a synthetic receive aperture by acquiring different subsets of the full aperture on successive transmit events. Multiplexing may also provide for the grouping of elements by connecting adjacent elements on either transmit or receive. Grouping by different factors is also possible such as, for example, using a group of three elements on transmit and a group of two elements on receive. One embodiment may provide multiplexing for synthetic transmit focusing mode and multiplexing for acoustic transmit focusing mode and provide for switching from one mode to the other, for example, on frame boundaries. Other multiplexing schemes are also possible and are contemplated by the disclosed embodiments.

Multiplexing may be controlled by using transmit timing, TGC and multiplexer control 212. Various transmit and/or receive elements may be selected when imaging a particular spatial region. For example, ultrasound echo data for an image frame may be acquired by sequentially interrogating adjacent sub-regions of a patient's body until data for the entire image frame has been acquired. In such a case, different sub-apertures (which may include elements numbering less than the full array) may be used for some or all sub-regions. The multiplexer control function may be programmed to select the appropriate sub-aperture (transmit and/or receive), for example, for each transmit excitation and each image region. The multiplexer control function may also provide control of element grouping.

Analog to Digital (A/D) converter 218 may convert the analog image data received from probe 100 into digital data using any method. Digital demodulator 222 may include any type of digital complex mixer, low-pass filter and re-sampler after each A/D converter channel, for example. In some embodiments, the digital mixer may modulate the received image data to a frequency other than a center frequency of probe 100. It some embodiments, this function may be performed digitally rather than in the analog or sampling domains to provide optimum flexibility and minimal analog circuit complexity. The low-pass filter may reduce the signal bandwidth after mixing and before re-sampling when a lower sampling rate is desired. One embodiment may use quadrature sampling at A/D converter 218 and, therefore, such an embodiment may not require a quadrature mixer to translate the digital data (e.g., radio frequency (RF)) signals of transducer 102 to a baseband frequency. However, complex demodulation by means of an analog or digital mixer or the like may also be used in connection with an embodiment.

Memory buffer 224 may have sufficient storage capacity to store up to, for example, two frames of data. Such a frame-sized buffer 224 may allow frames to be acquired at a rate substantially higher than the rate at which frames can be transferred to main unit 130 (or some other device) across wireless interface 120, for example. Such a configuration may, in an embodiment, be preferable to acquiring each frame over a longer time interval because a longer time interval may reduce a coherence of the acquired data throughout the frame. If frame transmission rates are at least as fast as frame acquisition rates, a smaller memory buffer 224 may be used in some embodiments. One embodiment uses a “ping-pong” buffer fed by the receiver channels as memory buffer 224. Data from multiple channels may be time interleaved into memory buffer 224. For example, 32 receiver channels each sampled at the rate of 6 MHz would produce a total baseband data rate of 192 M words per second, which is well within the rates of conventional DDR2 SDRAM. The ping-pong nature of memory buffer 224 may allow new data to fill buffer 224 while previously acquired data is read from memory and sent to wireless interface 120, for example.

Memory buffer 224 is followed by data merger 226. Data merger 226 may operate to merge receive channel data into one or more data streams before advancing the data stream to wireless interface 120 for transmission to main unit 130, for example. Data from data merger 226 may be sent across wireless interface 120 (and/or across wired interface 122) at a rate that is appropriate for the transmission medium. The data from the receive channels may be multiplexed in some fashion prior to transmission over wireless interface 120 and/or wired interface 122. For example, time-division multiplexing (TDM) may be used. Other types of multiplexing are also possible such as, for example, frequency-division multiplexing (FDM), code-division multiplexing (CDM), and/or some combination of these or other multiplexing techniques.

In addition to image data transfer, control information may be transferred between probe 100 and main unit 130. Such control data may be transferred over the same communication link, such as wireless interface 120 and/or wired interface 122, or some other communication link. Control commands may be communicated between main unit 130 and probe 100 (and/or some other devices). Such control commands may serve various purposes, including for example, instructing a mode of operation and/or various imaging parameters such as maximum imaging depth, sampling rate, element multiplexing configuration, etc. Also, control commands may be communicated between probe 100 and main unit 130 to communicate probe-based user controls 104 (e.g., button pushes) and probe operational status (e.g., battery level from power supply management 230), and the like.

The probe's status may include an indicator and/or display of certain values relevant to the operation of the system. For example, the indicator may be visible, audio, and/or some combination thereof. Without limitation, the indicator may indicate power status, designation of device, type of device, frequency range, array configuration, power warnings, capability of a remote unit, quality of transmission of digital data, quantity of errors in transmission of digital data, availability of power required for transmission of digital data, change in transmission rate, completion of transmission, quality of data transmission, look-up tables, programming code for field programmable gate arrays and microcontrollers, transmission characteristics of the non-beamformed ultrasound wave, processing characteristics of the echoed ultrasound wave, processing characteristics of the digital data, and/or transmission characteristics of the digital data, etc. Also, the indicator may show characteristics of a power source like capacity, type, charge state, power state, and age of power source.

In some embodiments, data/control arbiter 228 may be responsible for merging control information and image data communicated between probe 100 and main unit 130. The control information may be passed from control interface 232, where it is collected to data/control arbiter 228 for transmission to main unit 130. In some embodiments, control and image data may be distinguishable from each other when sent across wireless interface 120 and/or wired interface 122 to facilitate proper handling at main unit 130. In other embodiments, there may be no such distinction. In addition, data/control arbiter 228 may accept control commands from main unit 130 (and/or another device) and respond by appropriate programming of probe 100 circuitry, memory-based tables, registers, etc.

It will be appreciated that in an embodiment where probe 100 is to be used in a sterile environment, the use of wireless interface 120 to main unit 130 may be desirable, as the use of wireless interface 120 avoids many of the problems associated with having a physical connection between probe 100 and main unit 130 that passes into and out of a sterile field. In other embodiments, certain sheathing or sterilization techniques may eliminate or reduce such concerns. In an embodiment where wireless interface 120 is used, controls 104 may be capable of being made sterile so as to enable a treatment provider to use controls 104 while performing ultrasound imaging tasks or the like. However, either wireless interface 120 or wired interface 122, or a combination of both, may be used in connection with an embodiment.

Probe 100 circuitry also includes power supply 236, which may operate to provide drive voltage to the transmitters as well as power to other probe electronics. Power supply 236 may be any type of electrical power storage mechanism, such as one or more batteries or other devices. In one embodiment, power supply 236 may be capable of providing approximately 100V DC under typical transmitter load conditions. Power supply 236 also may also be designed to be small and light enough to fit inside a housing of probe 100, if configured to be hand held by a treatment provider or the like. In addition, power supply management circuitry 230 may also be provided to manage the power provided by power supply 236 to the ultrasound-related circuits of probe 100. Such management functions include monitoring of voltage status and alerts of low-voltage conditions, for example.

Controls 104 may be provided to control probe 100. Control interface 232 may pass user input received from controls 104 to data/control arbiter 228 for processing and action, if necessary. Such control information may also be sent to the main unit 130 through either wireless interface 120 and/or wired interface 122. In addition to sending data to main unit 130, wireless interface 120 may also receive control or other information from main unit 130. This information may include, for example, image acquisition parameters, look-up tables and programming code for field programmable gate arrays (FPGAs) or microcontrollers residing in probe 100, or the like. Controller interface 232 within probe 100 may accept and interpret commands from main unit 130 and configure probe 100 circuitry accordingly.

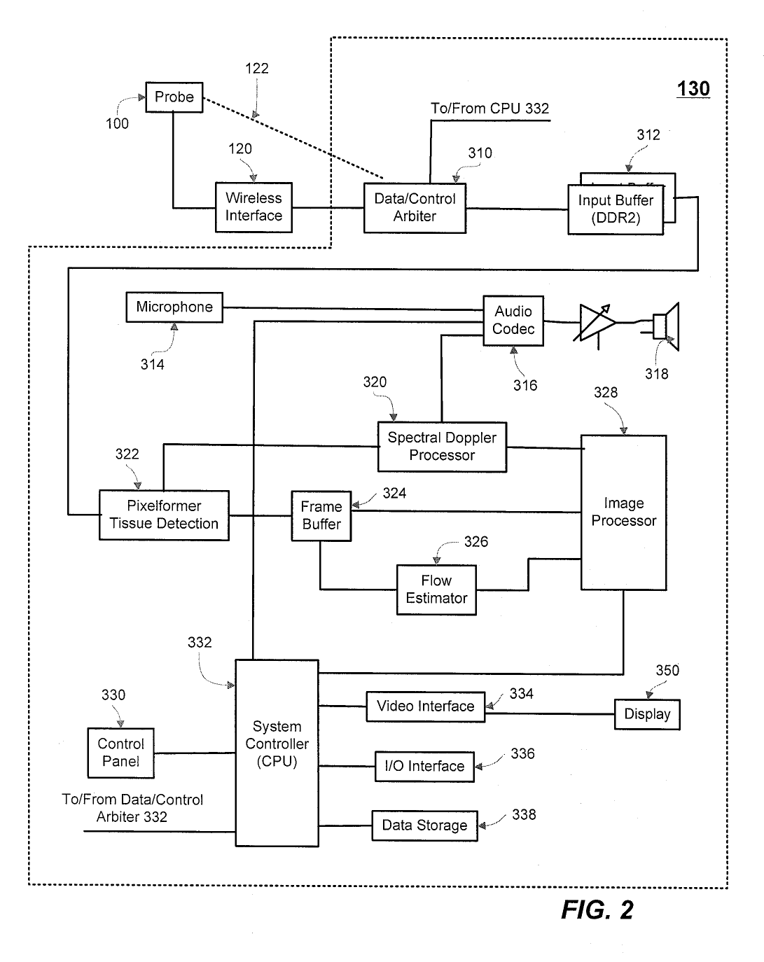

Now that an example configuration of components of probe 100 has been described, an example configuration of components of main unit 130 will be discussed with reference to FIG. 2. It should be noted that any or all of the components illustrated in FIG. 2 may be disposed within one or more housings (not shown in FIG. 2) having any form factor.

As discussed above, probe 100 may be in communication with main unit 130 by way of wireless interface 120 and/or wired interface 122. It will be appreciated that in an embodiment most data transfer occurs from probe 100 to main unit 130, although in some embodiments more data may be transferred from main unit 130 to probe 100. That is, large amounts of image data sent from probe 100 may be received by main unit 130, as well as control information or the like. Control information is managed and, in many cases, generated by Central Processing Unit (CPU) controller 332. CPU controller 332 may also be responsible for configuring circuitry of main unit 130 for an active mode of operation with required setup parameters.

In some embodiments, data/control arbiter 310 may be responsible for extracting control information from the data stream received by wireless interface 120 and/or wired interface 122 and passing it to CPU 332 while sending image data from the data stream to input buffer 312. Data/control arbiter 310 may also receive control information from CPU 332, and may transfer the control information to probe 100 via wireless interface 120 and/or wired interface 122.

A user, such as a treatment provider or the like, may control the operations of main unit 130 using control panel 330. Control panel 330 may include any type of input or output device, such as knobs, pushbuttons, a keyboard, mouse, and/or trackball, etc. Main unit 130 may be powered by any type of power supply (not shown in FIG. 2) such as, for example, a 120 VAC outlet along with AC-DC converter module, and/or a battery, etc.

To facilitate forming an image on display 350 (e.g., pixelforming—a process that generates an ultrasound image from the image data received from probe 100), the incoming image data may be stored in input buffer 312. In an embodiment, input buffer 312 may be capable of storing up to approximately two frames of data, for example, and may operate in a “ping-pong” fashion whereby a previously received frame of data is processed by pixelformer 322 while a new incoming frame is written to another page of memory in input buffer 312. Pixelformer 322 may be any combination of hardware and/or software that is capable of transforming raw image data received from the receive channels and the transmit events (e.g., from probe 100) into a pixel-based image format. This may be performed, in just one example, by coherently combining data from various transmit and receive elements, or groups of elements, to form an image focused optimally at each pixel. Many variations of this approach may be used in connection with an embodiment. Also, this function may include a beamformer that focuses samples along beam directions. The focused sample data may be converted to a Cartesian format for display on display 350.

Once a frame of complex pixel data has been formed, it may be stored in frame buffer 324 for use by either flow estimator 326 and/or image processor 328. In an embodiment, flow estimator 326 uses, for example, Doppler or cross-correlation methods to determine one or more flow characteristics from the received image (e.g., ultrasound echo) data. Once the flow estimation parameters have been computed, they may be encoded into data values and either stored in frame buffer 324 for access by image processor 328 and/or sent directly to image processor 328. Note that the term “pixel” as used herein typically refers to an image sample, residing on a Cartesian polar and/or non-uniform coordinate grid, computed by processing captured echo signal data. Actual display pixels may differ from these image pixels in various ways. For example, the display pixels, as presented on display 350, may be a scaled, resized, filtered, enhanced, or otherwise modified version of the image pixels referred to herein. These functions may be performed by a processor, for example, image processor 328. Pixel also may refer to any finite level, value, or subcomponent of an image. For example, an image that is made up of a number of subcomponents, both visual and otherwise, may be referred to as a pixel.

Spectral Doppler processor (SDP) 320 may receive focused baseband data from pixelformer 322 from one or more spatial locations within the image region in a periodic or other fashion. The spatial locations may be referred to as range gates. SDP 320 may perform high-pass filtering on the data to remove signal contributions from slow moving tissue or the like. The remaining higher frequency signals from blood flow may be in the normal audio frequency range and these signals may be conventionally presented as an audible signal by speaker 318. Such audio information may, for example, assist a treatment provider in discerning a nerve from a blood vessel and/or a vein from an artery. SDP 320 may also perform spectral analysis via a discrete Fourier transform computation, or other means, to create an image representing a continuously updated flow velocity display (i.e., a time-varying spectrogram of the blood flow signal). The velocity data may be sent through image processor 328 for further processing and display.

A user of main unit 130 may use microphone 314 for controlling main unit 130 using, for example, voice recognition technology. Alternately, or in addition to using microphone 314 for control purposes, a user may use microphone 314 for taking notes while examining a patient. Audio notes may be saved separate from, or along with, video data.

Audio codec 316 may accept audio data input from microphone 314 and may interface with CPU 332 so audio data received by audio codec 316 may be stored and/or interpreted by CPU 332. Such audio interpretation may facilitate system control by way of, for example, voice commands from a user of main unit 130. For example, frequently-used system commands may be made available via voice control. Such commands may also be made available by way of control panel 330, for example. Audio storage facilitates audio annotation of studies for recording patient information, physician notes and the like. The audio data may first be converted to a compressed format such as MP3 before storing in, for example, storage 338. Other standard, proprietary, compressed or uncompressed formats may also be used in connection with an embodiment. Speaker 318 may provide audio output for reviewing stored annotation or for user prompts from main unit 130 resulting from error conditions, warnings, notifications, etc. As mentioned above, Doppler signals may also be output to speaker 318 for user guidance in range gate and/or steering line placement and vessel identification.

Video interface 334 may be in communication with image processor 328 to display 350 by way of CPU 332. Display 350 may be any device that is capable of presenting visual information to a user of main unit 130 such as, for example, an LCD flat panel, CRT monitor, composite video display or the like. Video data may also be sent to storage 338, which may be a VCR, disk drive, USB drive, CD-ROM, DVD or other storage device. Prior to storage, for example, still image frames of data may be encoded in a compressed format such as JPEG, JPEG2000 or the like. Image clips or sequences may be encoded in a format such as MJPEG, MJPEG2000 or a format that includes temporal compression such as MPEG. Other standard or proprietary formats may be used as well.

Image processor 328 may accept either complex and/or detected tissue image data and then filter it temporally (i.e., frame to frame) and spatially to enhance image quality by improving contrast resolution (e.g., by reducing acoustic speckle artifact) and by improving SNR (e.g., by removing random noise). Image processor 328 may also receive flow data and merge it with such tissue data to create a resultant image containing both tissue and flow information. To accomplish this, image processor 328 may use an arbitration process to determine whether each pixel includes flow information or tissue information. Tissue and/or flow pixels may also be resized and/or rescaled to fit different pixel grid dimensions either prior to and/or after arbitration. Pixels may also be overwritten by graphical or textual information. In an embodiment, both the flow arbitration and graphical overlay may occur just prior to image display to allow the tissue and flow images to be processed independently.

Temporal filtering typically may be performed on both the tissue and flow data prior to merging the data. Temporal filtering can yield significant improvements in SNR and contrast resolution of the tissue image and reduced variance of the flow image while still achieving a final displayed temporal resolution suitable for clinical diagnosis. As a result, relatively higher levels of synthetic aperture subsampling may be provided, thereby reducing the required and/or desired number of receiver channels (and, consequently, in some embodiments power consumption of probe 100). Temporal filtering typically involves filtering data from frame to frame using either an FIR or IIR-type filter. In one embodiment, a simple frame averaging method may be used as discussed below, for example.

Temporal filtering and/or persistence is commonly applied to frames of ultrasound data derived from, for example, tissue echoes. When the acquisition frame rate exceeds the rate of motion of anatomical structures, low-pass filtering across frames can reduce random additive noise while preserving or enhancing image structures. Also, minute degrees of motion—commonly due to patient or operator movement—help to reduce image speckle, which is caused by the interference of acoustic energy from randomly distributed scatterers that are too small to be resolved with the frequency range of ultrasound probe 100. Speckle is coherent by its nature so, in the absence of motion, it may produce the same pseudo-random noise pattern on each image frame. However, small amounts of motion diversify the speckle enough to make low-pass filtering across frames effective at reducing it.

A simple method of temporal filtering may involve averaging neighboring frames. An example of the recursive version of a moving-average filter is described as follows where X(n) is the input frame acquired at time n, Y(n) is the corresponding output frame, and k is a frame delay factor that sets the size of the averaging window:

Y(n)=Y(n−1)+X(n)−X(n−k) (1)

Another simple low-pass filter is a first-order IIR filter of the form:

Y(n)=C×Y(n−1)+(1−C)×X(n) (2)

In such an embodiment, the coefficient C sets the filter's time constant and the degree of low-pass filtering applied to the frame sequence. It should be appreciated that Equations (1) and (2) are just examples of possible filters and filtering techniques that may be used in connection with an embodiment.

Control panel 330 may provide pushbuttons, knobs, etc., to allow the user to interact with the system by changing modes, adjusting imaging parameters, and so forth. Control panel 330 may be operatively connected to CPU 332 by way of, for example, a simple low bandwidth serial interface or the like. Main unit 130 may also include one or more I/O interfaces 336 for communication with other devices, computers, a network or the like by way of a computer interface such as USB, USB2, Ethernet or WiFi wireless networking, for example. Such interfaces allow image data or reports to be transferred to a computer or external storage device (e.g., disk drive, CD-ROM or DVD drive, USB drive, flash memory, etc.) for later review or archiving, and may allow an external computer or user to control main unit 130 remotely.

There are at least two techniques used for interrogating a medium and processing the data needed to create an ultrasound image: synthetic transmit focusing and acoustic transmit focusing. In synthetic transmit focusing, the interrogating ultrasound waves may be transmitted into the medium, from various locations in the array, in an unfocused or defocused manner, and reflected waves are received and processed. Somewhat differently, with acoustic transmit focusing the interrogating ultrasound waves may be transmitted in a way that provides focus at certain spatial locations in the medium, and therefore the transmitted ultrasound wave cooperates to form a “beam.” Various embodiments contemplate synthetic transmit focusing, acoustic transmit focusing, and/or a combination of both. One embodiment contemplates dynamically switching between synthetic transmit focusing and acoustic transmit focusing modes periodically. For example, color flow data acquisition may use acoustic transmit focusing while tissue imaging may use synthetic transmit focusing. Color flow and tissue data may be collected on some alternating basis, for example. Other embodiments may include the use of non-beamformed techniques, in which, a beam may not be formed and/or be partially formed. Similarly, these beamformed and non-beamformed techniques may be used after the medium is interrogated in evaluating the echoed ultrasound waves and/or the digital data from which these waves are formed.

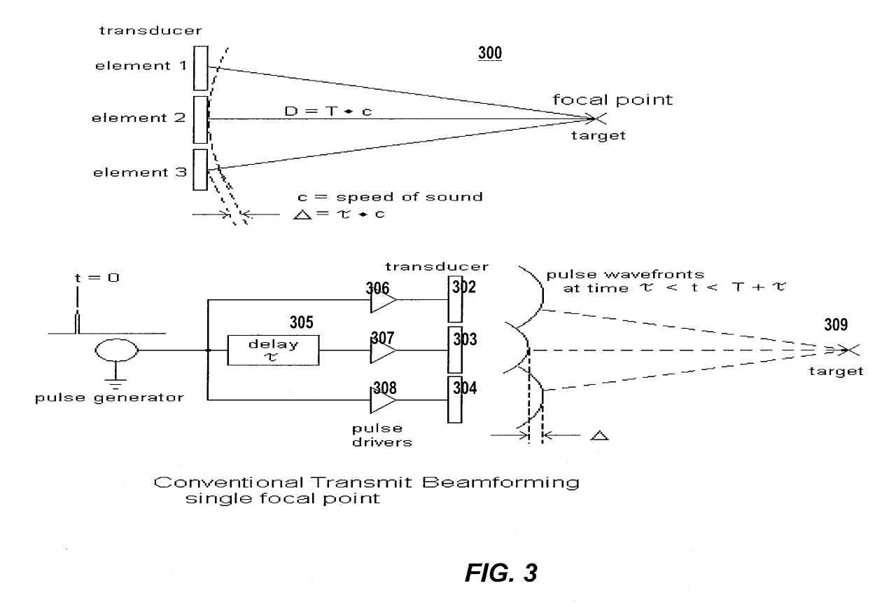

FIG. 3 is a block diagram of a system 300 for transmitting an acoustic transmit focusing wave. As shown in FIG. 3, a pulse generator 301 provides a signal to a transducer element 302, a transducer element 303, and a transducer element 304. The signal provided by pulse generator 301 to transducer element 303 may be provided via a delay module 305. Although not shown in FIG. 3, it should be appreciated that other delay modules may be provided between other transducers. Also, although just three transducers are shown in FIG. 3, it should be appreciated that many other transducer and arrays of transducers are contemplated in the embodiments.

Each of the transducers may receive the signal via a respective pulse driver. For example, a pulse driver 306 may be in communication with transducer element 302, a pulse driver 307 may be in communication with transducer element 303, and a pulse driver 308 may be in communication with transducer element 304. The transducers may be acoustic transducers that convert the signal provided by pulse generator 301 from an electrical signal to an acoustic and/or ultrasonic wave. In some embodiments, the size (physical or electrical) of the transducer elements may be sufficiently small to allow the transducer elements to effectively act as point radiators in a predetermined frequency range. The timing of the pulses provided to the transducers and thus the timing of the acoustic waves created by the transducers may be of any nature, according to the contemplated embodiments. For example, the arrangement may be a phased array whereby transmit focal points are typically located at equal radial distances from a common vertex. The transmit beams are usually located at equal angular distances from each other and may span a total of 90 degrees or more. While the transmit focus is typically located at one point along the beam, echo data is usually collected along the entire beam length starting at the vertex and ending at a point corresponding to some maximum imaging depth. At radial locations other than the transmit focal point, the transmit beam diverges with the beam becoming increasingly unfocused at radial locations furthest from the focal point.

The acoustic waves created by the transducers interrogate a particular point or target 309 within a medium. Target 309 may be of any size or dimension. In some embodiments, target 309 may be considered to be a point-reflector, such that its dimensions are relatively small compared to the wavelength of the ultrasound wave. In this embodiment, the target may be considered to effectively be a Dirac delta function in space, such that the reflected echo wave provides a substantial replica of the wave that hits and interrogates target 309.

In just one example, target 309 is some distance “D” from a center line of transducer element 303. With “c” as the speed of sound, the amount of time it takes an ultrasound wave to travel from transducer element 303 to target 309 is calculated as T=D/c. The distance from transducer element 304 to target 309 is D+Δ, so Δ is the difference between transducer element 304 distance to target 309 and transducer element 303 distance to target 309. The time it takes to travel the distance Δ is τ=Δ/c.

In some embodiments, it may be desirable to apply delays between the pulse generator signals and transducer elements for some purpose. For example, in one embodiment, it may be desirable to provide delays to create a more focused wavefront at a particular point, like target 309. In a focused wavefront, the ultrasound waves generated by each transmitting transducer element may sum substantially constructively at one location within the field of view (FOV) and relatively destructively at the other locations in the FOV. In this example, it may be that transducer elements 302 and 304 create their ultrasound waves first in time, followed by transducer element 303 at a time τ later. FIG. 3 captures an example of the emitted acoustic waves some time later, for example, t<T+τ. These waves created by the transducers will converge and constructively interfere at the focal location, creating a pressure wave that is the coherent sum of the three transmit waves. The waves will all arrive at the focal point at time t=T+τ. Typically, under normal conditions, at the other points in space, the waves will not constructively sum.

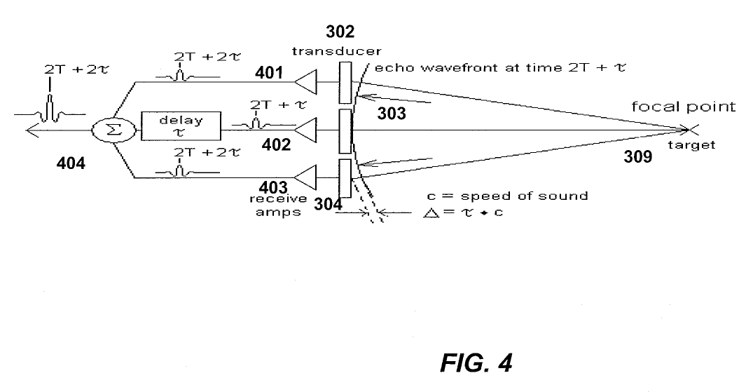

FIG. 4 is a block diagram of a receive beamformer system. As shown in FIG. 4, target 309 reflects the transmitted ultrasound wave back to transducers 302-304. Although transducers 302-304 are shown as being the same as the transducers that transmitted the interrogating ultrasound wave, the embodiments are not so limited. Instead, it should be appreciated that the echo wave may be received by any available transducers, including only a portion of the transmitting transducers and/or different transducers. Any combination thereof is contemplated.

As shown in FIG. 4, target 309 reflects at least a portion of the transmitted ultrasound wave back to the transducers. As a result of the smaller target dimensions, in this example, the reflected wave is substantially hemispherical. Although FIG. 4 illustrates the echo waves as sinusoidal pulses (typical of ultrasound transducers), it should be appreciated that the echo waves contemplated by the embodiments may be of any characteristic. Also, it should be appreciated that the echo waves may have any type of characteristic frequency F_{c}, that may be modulated with an envelope that may be modeled as Gaussian and/or other windowing function. For example, where F_{bw} is the bandwidth of the modulation envelope, a fractional bandwidth, F_{bw}/F_{c} may be 50% to 70% (at the −6 dB points) for typical transducers.

In this example, at a time 2 T+τ, the reflected acoustic wave reaches transducer element 303. The transducers act to convert the acoustic wave into electrical energy. Transducer element 303 may provide the electrical energy signal to an amplifier 402 that amplifiers the electrical energy signal as required by the remainder of the system. At a later time, for example, 2 T+2τ, the reflected wave reaches transducer elements 302 and 304.

Transducer elements 302 and 304 convert the acoustic wave into electrical energy that is amplified, respectively, by amplifiers 401 and 403. The electrical energy provided by the transducers may be either analog or digital signals. Also, the analog electrical signals may be analog and later converted to digital signals, for example, using analog-to-digital (A/D) converters (not shown). Such conversion to digital signals may be accomplished at any point in the system, as contemplated by the embodiments. Time delay 305 causes a delay in the electrical signal from amplifier 402, such that the electrical signals from the three amplifiers arrive at a summer 404 substantially simultaneously, or at least in close enough proximity of time to allow the signals to sum constructively. Such time delay may be accomplished on both analog and digital electrical signals.

Summer 404 adds the three electrical signals, and the summed signal is transmitted to further circuitry (not shown) for further processing and analysis. For example, in just one embodiment, the summed signal may have its magnitude, amplitude and/or phase sent to a processor who determines the corresponding values and converts the values into an image value (e.g., brightness). B-Mode typically refers to determining an image's brightness value based on the amplitude of the summed echo signals near a transmitted center frequency.

Another method for interrogating a medium and processing the data needed to create an ultrasound image involves synthetic transmit focusing. With synthetic transmit focusing methods, each pixel of an image may be formed from data acquired by multiple transmit events from various locations of the transducers. Generally, with synthetic transmit focusing, sequentially acquired data sets may be combined to form a resultant image.

On the transmit side of a synthetic transmit focusing system, it may be desirable to interrogate as broad an area of the medium as possible. Broad interrogation may be accomplished using many techniques.

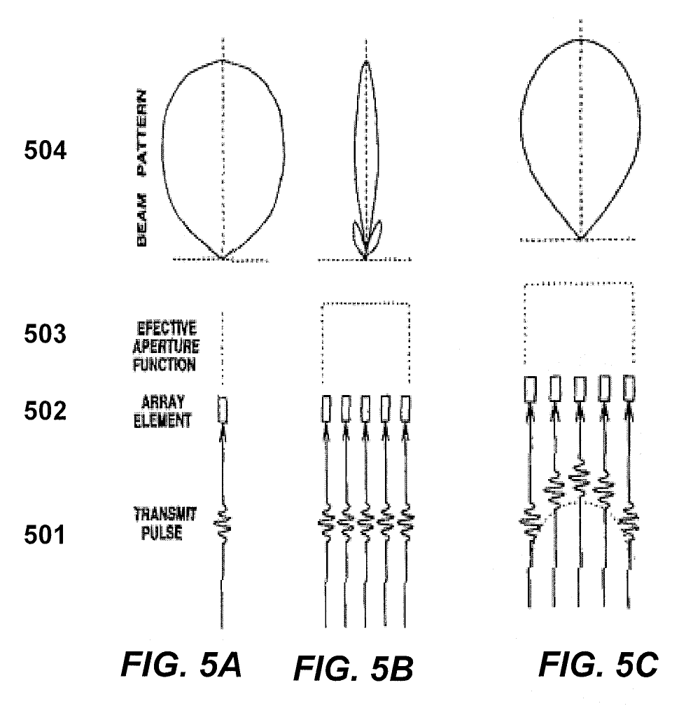

FIGS. 5A-5C illustrate examples of different possible configurations and techniques for providing such interrogation using different transducers and transducer configurations. In particular, FIGS. 5A-5C provide examples of a transmit pulse or pulses 501, an arrangement or array of transducer elements 502, an effective aperture 503, and a resultant beam pattern 504. For example, as shown in FIG. 5A, sequentially providing transmit pulses a single transducer element (for example of an array of transducers) may create a broad beam pattern. Another example shown in FIG. 5B illustrates providing a series of transmit pulses each to an individual transducer at substantially the same time. Finally, as shown in FIG. 5C, providing a transmit pulse to each transducer in a certain sequence may also create a broad beam pattern. FIG. 5C provides just one example of a defocused transmit, which may permit greater signal-to-noise ratio (SNR) and better sensitivity off the center line of the transducer elements. Although the beam pattern created by FIG. 5B may not be as broad as the example in FIG. 5A or 5C, it may be sufficient in certain contemplated embodiments.

As shown in FIG. 5, a single transducer or transducer configuration is sufficient for a single investigation type. When the need arises for a different investigation type, such as a deep tissue probe as opposed to a probe through a bone structure such as a rib cage, a different transducer must be selected. While wide-band probes partially address the need for multiple transducer types, they are less than ideal. First, wide-band transducers are, generally, more costly to manufacture than low bandwidth transducers. Also, in order to drive the transducer at the edges of its frequency band, the system transmitter electronics must be fairly sophisticated resulting in a higher system cost. Also, the large operating frequency of wide-band probes does nothing to address the other factors that users require such as array dimensions and curvature to make the appropriate tradeoffs between penetration, far field focus, and field of view. Consequently, users often purchase and operate several different transducer types with most commercially available ultrasound systems.

In an exemplary embodiment, a users need to cover a range of imaging application is met by a range of transducers that may consist of two or more different transducers in one probe housing in order to span a broader range of applications with one probe. This could be less expensive and more convenient than providing two or more separate probe assemblies, as is the current convention. In addition, the single probe may be configured with a wireless transducer for data transmission. With a wireless transducer, there is no cable, so there is the potential to provide a single probe assembly with two different transducers, one on each end. The ergonomics of doing this are more manageable if there is no cable attached to the housing. In additional embodiments, other physical configurations could be considered.

In exemplary embodiments, the various characteristic transducer types that may be supported include, but are not limited to, linear arrays, phased arrays, and curvilinear arrays. Different frequency ranges may also be offered on the same probe assembly with either the same or different transducer types.

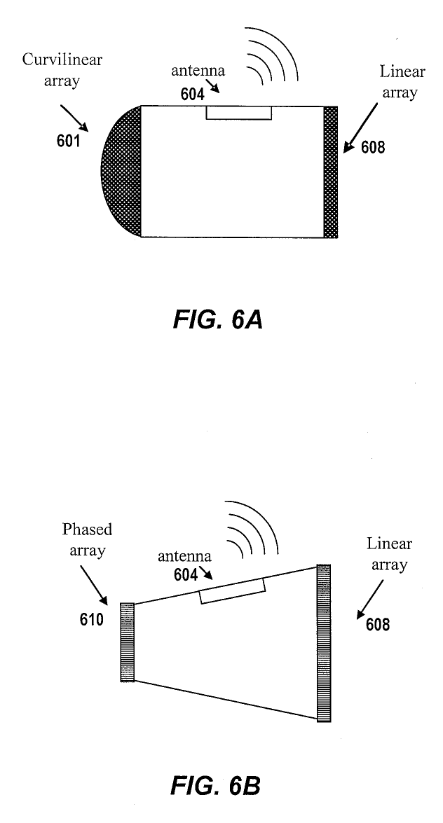

Regarding FIG. 6A, this figure illustrates an exemplary combination of linear 608 and curvilinear array 601 transducers. The linear array 608 may, for example, have a higher center frequency for near-field vascular imaging while the curved array 601 may be a lower frequency transducer designed for deeper abdominal imaging. As seen in the embodiment represented by FIG. 6A, the probe is an enlongated shape with a curvilinear array 601 configured at one end of the single probe, and the linear array 608 configured at the opposite end of the single probe. Additionally, in this exemplary embodiment, for efficient operation of the data transmission from the probe to the main unit of the ultrasound system, a wireless antenna 604 may be configured in the middle section of the single probe. The antenna 604 being configured to wirelessly transmit the data collected from either the curvilinear array 601 or the linear array 608 as the data is collected during probe operation.

Regarding FIG. 6B illustrates an exemplary embodiment of a phased array 610 and linear array 614 transducers within a single probe. In this exemplary embodiment, the linear array 614 may, again, have a higher center frequency for near-field vascular imaging while the phased array 610 may be a lower frequency transducer designed for cardiac imaging where access through the rib cage is required. As seen in the embodiment represented by FIG. 6B, the probe is an enlongated shape with a phased array 610 configured at one end of the single probe, and the linear array 608 configured at the opposite end of the single probe. Additionally, in this exemplary embodiment, for efficient operation of the data transmission from the probe to the main unit of the ultrasound system, a wireless antenna 604 may be configured in the middle section of the single probe. The antenna 604 being configured to wirelessly transmit the data collected from either the curvilinear array 601 or the linear array 608 as the data is collected during probe operation.

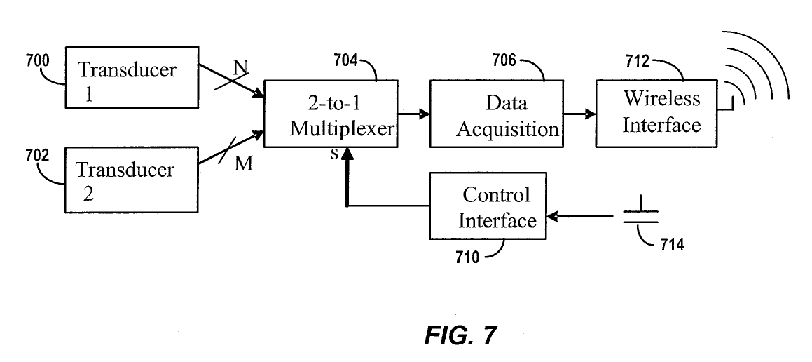

Regarding FIG. 7, this Figure provides an illustration of an exemplary embodiment of this invention in which each transducer is selectable in some way. Selection may be activated either by a control, such as a button, on the probe or the system's Main Unit. To implement this embodiment, high-voltage, electronic switches 714 or multiplexers 704 must be provided. Transducer 1 700 with N elements and Transducer 2 702 with M elements are represented as the Transducers configured within a single probe. In the exemplary embodiment, the switch 714 may be a multi-throw switch designed for use in a medical environment and configured to control power and provide on/off selection for all elements within the probe. In an exemplary embodiment a switch of the type HV209, supplied by Supertex, may be utilized, however, other suitable devices may also be used. The switch 714 is in electrical contact with the control interface 710 within the probe such that the switch may control the power on/off function of the probe, as well as the selection of the transducer to be used for each operation of the probe. When the probe is in operation, in this exemplary embodiment, the data collected by the selected transducer is transmitted to a 2-to-1 multiplexer 704 prior to being relayed to the Data Acquisition 706 circuit within the probe. The Data Acquisition 706 component prepares the data signal for transmission and relays the data signal to the Wireless Interface 712 component. The data is then transmitted over a wireless channel to the main unit of the ultrasound system.

In an alternative embodiment, one or more of the transducer heads may be removable to allow interchanging of different probe types. In this exemplary embodiment, a removable transducer head would require a high-density connector to facilitate the removal and reconnection of different transducer types. This high-density connector would add considerable cost and require considerable space within the single probe housing. In addition, the transducer-to-probe connection would be difficult to seal from ingress of coupling gel or other fluids. For these reasons, this configuration is less desirable.

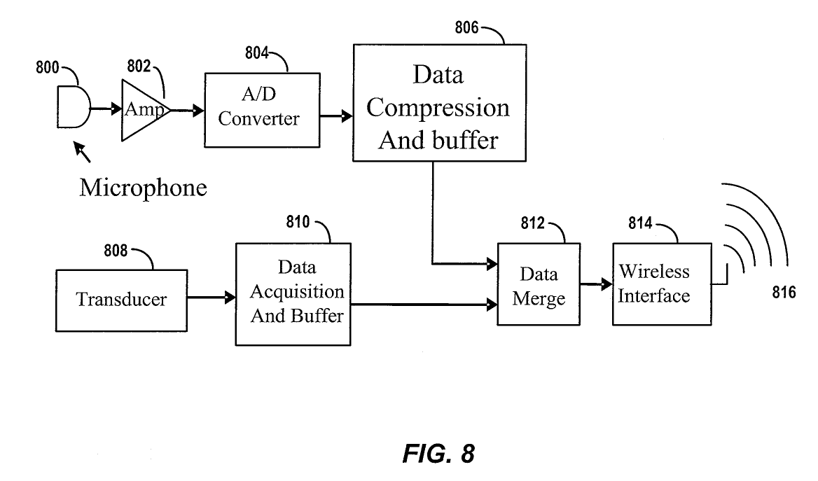

With regard to FIG. 8, this Figure provides an exemplary embodiment for other adjunct uses for the probe housing. In this exemplary embodiment, instead of providing a second transducer in the same housing, one exemplary adjunct use would be to provide a stethoscope in the same probe housing as the transducer 808.

In this exemplary embodiment, the stethoscope's acoustic signal is picked up by a microphone 800 in the transducer housing where it is amplified 802 and the signal data digitized utilizing a D/A converter 804. In the exemplary embodiment, after digitizing, the microphone data may be further processed 806 to provide data compression or signal enhancement. In this embodiment and by way of example, the microphone audio data may be compressed into the standard MP3 format or some other standard or proprietary format. This data signal is then merged 812 with the data acquired from the operation of the transducer after that data signal has also been compressed and buffered 810. The compressed and merged data signals are then transmitted wirelessly 816 to the main ultrasound system over the same wireless link 814 used for ultrasound signal information. In an alternative embodiment, the digitized audio data may also be sent across the wireless link 814 in raw form if the wireless bandwidth supports the required data rate.

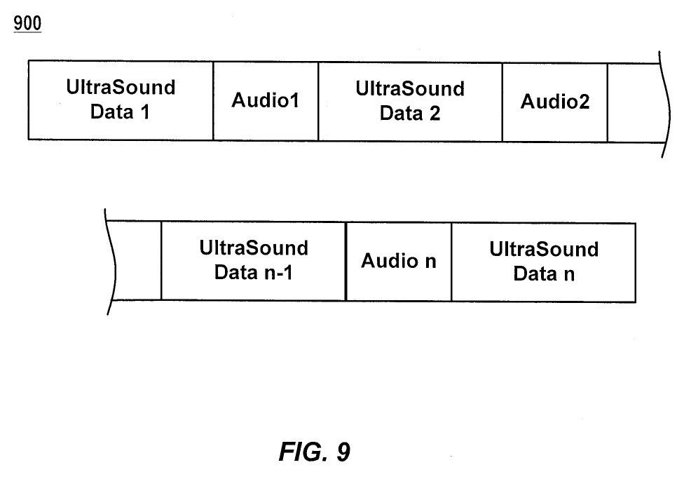

In this exemplary embodiment as shown in FIG. 9, to facilitate merging the audio and ultrasound acquisition data, both data streams are packetized and the packets multiplexed in some way. For example, the ultrasound data acquired on a single pulse transmission may be formed into a single packet with all transducer element signals time-multiplexed into a single data sequence. The microphone acquired data may be packetized according to an arbitrary time interval. The audio data packets are then merged with the ultrasound data packets by interleaving the audio data with one or more ultrasound data packets. Both the ultrasound and audio acquisition paths require some data buffering to facilitate the data packetization and interleaving of packets.

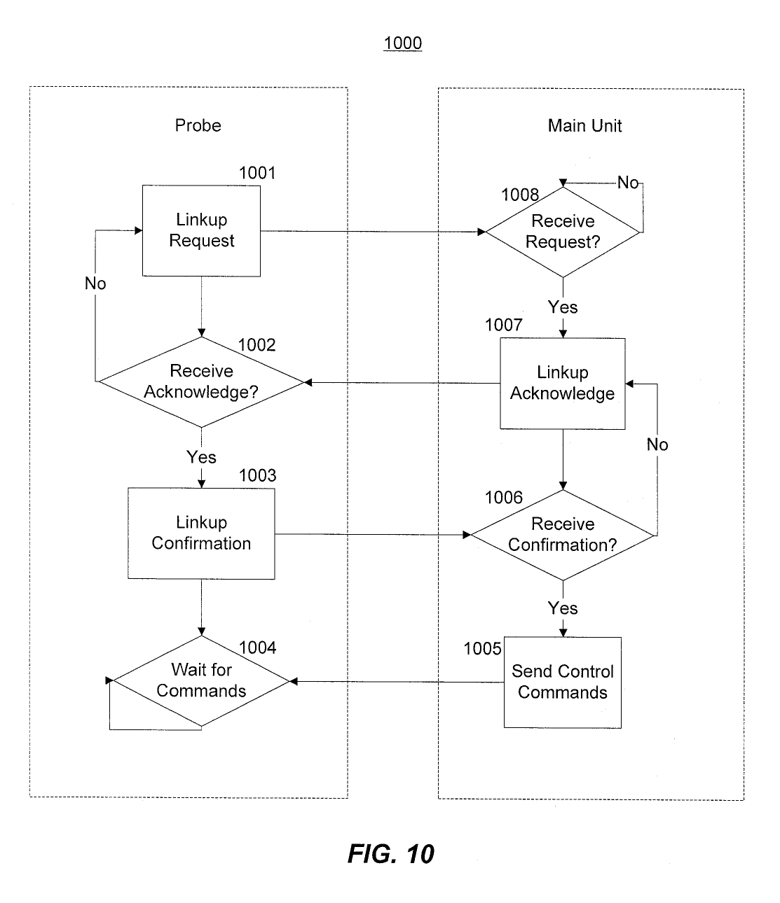

FIG. 10 is a flow diagram of a method 1000 for establishing a link between a probe and a main unit. It should be appreciated that although the method includes just the probe and the main unit, the link may involve other components and processes. Also, the embodiments contemplate other methods for establishing such a link.

The primary and/or alternate channels also may be used to sense a proximity of the main unit from the probe and vice versa. For example, in some embodiments, the primary and/or alternate channels may employ IR, capacitive, inductive, magnetic, and/or any other technique commonly used in sensing a proximity of one device from another.

Proximity sensing may be employed for a variety of purposes, all of which are contemplated by the disclosed embodiments. For example, in some embodiments, it may be desirable to establish an exclusive link between a particular probe and a particular main unit based on a proximity between the two and/or between other devices. Since determining proximity may be difficult using signal properties of a primary RF communication channel, for example, an alternate channel may be utilized in order to facilitate the linkup process. Some alternate communication channels described above (e.g., IR) may be highly directional while others may be specifically designed for proximity sensing. These channels may be used alone and/or in conjunction with another communication channel, for example, during the linkup process.

An exclusive link between probe and main unit may serve a variety of purposes including providing for interoperability of multiple wireless probe-based systems in close proximity to one another, for example. This characteristic of the exclusive link, in some embodiments, may include a temporal limitation. For example, it may be desirable to allow the exclusive link to endure for at least one operating session and/or over some predetermined period of time.

The exclusive link may be initiated by either the probe or the main unit or by some other means. For example, a user may press a button or the like located on the probe, main unit or other device. The exclusive link may be established by communicating a particular data sequence and/or particular data character between the main unit and the probe.

Also, the linkup process may allow the main unit and remote unit (or another unit) to distinguish and/or identify each other. For example, the distinction may be accomplished by determining a proximity of the main unit to the remote units, a relative strength of a signal communicated by the main unit with the remote units, a predetermined identifier, and/or an absence of the another remote unit. The predetermined identifier may include a registered identifier and/or an identifier used in a previous communication between the main unit and the remote units. This also may be accomplished through the use of control data that is unique to the main unit and the remote unit, where the control data initiates, synchronizes and/or ensures communication between the main unit and the remote unit. This communication may be facilitated by the use of one or more antennae located the main unit and the remote unit. The antennae may be arranged to prevent multipath effects including distortion and signal nulls.

In one embodiment, as shown in FIG. 10, for example, the probe may initiate communication with a nearby main unit by transmitting a “linkup request” command at 1001 over the wireless communication channel, for example. At 1008, it is determined whether the main unit has received the linkup request. If the main unit has not received the linkup request, the main unit continues at 1008 to wait for the linkup request. If the main unit has received the linkup request, in some embodiments, the main unit may respond with a “linkup acknowledge” command at 1007 sent back to the probe. This “linkup acknowledge” command may provide information relevant to the communication. For example, the “linkup acknowledge” may indicate that the probe is within sufficient range of the main unit to permit wireless communication. Also, the proximity sensing and linkup communication may allow either the probe and/or main unit to automatically wake up from a low-power state, standby mode, and/or otherwise change power status.

At 1002, the probe determines whether is has received the linkup acknowledge. If the probe has not received the linkup acknowledge, the method may return to 1001 to wait for another linkup request. This return may occur after a predetermined condition, like a timeout or another predetermined period of time.

If the probe has received the linkup acknowledge, in some embodiments, at 1003 the probe may communicate back to the main unit with a “linkup confirmation” command to indicate that the communication is established. At 1006, the main unit may determine if it has received the linkup confirmation. If the main unit has not received the linkup confirmation the method may return to 1007 to wait for another linkup acknowledge. This return may occur after a predetermined condition, like a timeout or another predetermined period of time. If the main unit has received the linkup confirmation, in some embodiments, at 1005 the main unit may communicate back to the main unit with a “linkup complete” command to indicate that the linkup is complete. Along with the linkup complete commands the main unit may provide control commands to the probe. At 1004, the probe may loop to wait for the commands.

It should be appreciated that the linkup commands may be initiated by either the probe, main unit, and/or some other device, and thus the particular commands may be sent by any of the devices. Also, it should be appreciated that additional communication and corresponding commands relevant to the linkup of the devices are contemplated by the disclosed embodiments. In addition, the linkup may be attempted a certain number of predefined times before it is ceased.

In order to facilitate the linkup process, in some embodiments, both the probe and main unit may be pre-assigned unique identifier codes or identification numbers (e.g., serial numbers), that may be communicated between the main unit and probe (and perhaps other devices) during the linkup process.

The identifier codes may allow, for example, subsequent exclusivity with respect to further communications between the probe and main unit and allow interoperability with multiple wireless probe-based systems in close proximity. It should be appreciated that in some embodiments, interoperability may be a consideration during the linkup process. For example, interoperability and exclusivity may be appropriate where there are multiple main units and/or probes or the like within the wireless communication range that may respond to the probe's and/or main unit's request. In some embodiments, it may be desirable to permit the probe and/or main unit that are in closest proximity to one another to linkup, while in other embodiments it may be appropriate to use other metrics (e.g., signal strength, power status and availability, use selection, most recently linked, etc.).

It should be appreciated that other techniques for accomplishing discrimination between the probe, main unit and/or other devices are contemplated within the disclosed embodiments. For example, non-wireless or wired communication techniques may be used in some embodiments. The techniques may include making electrical and/or magnetic contact between the probe and main unit and/or by allowing a user to press a button on the main unit.

It should be appreciated that the linkup process may be automatic or manual, or a combination of both. For example, some embodiments may permit the entire linkup process to occur without requiring the probe, operator or other device to make contact with the main unit. Other embodiments may require the user to initiate certain portions of the process manually. For example, the user may select a probe type from a displayed list of available probes resulting in the main unit sending a linkup request to probes of the selected type.

In some embodiments, it may be that after the linkup process has been completed, the probe and main unit may include some information (e.g., their identification numbers) in some or all subsequent communication. This may permit the devices to avoid subsequent conflicts or miscommunication with nearby systems. In addition, the probe and main unit may store their own and each other's identification numbers in order to facilitate subsequent linkups after a particular session is terminated or placed in a non-operative mode. For example, the identification numbers may be stored temporarily or permanently in non-volatile memory such as EEPROM, Flash PROM, or battery powered SRAM, well known to those skilled in the art. In this way, if the link between the probe and main unit link is terminated or discontinued for some period, either device (or another device) may attempt to reestablish the link. Such attempted reestablishment of the link may be accomplished automatically (e.g., periodically), upon some operator action, or based on some other input.

As shown in FIG. 2, the main unit may include or be in communication with a display unit. The display unit may display information about the main unit, a linked or other probe, and/or another device. With regard to the probe, the display may provide details regarding the probe type (e.g., frequency range, array configuration, etc.), an identifier code or number, a user pre-assigned name, etc. The name of the probe may be determined by the user and entered at the main unit, communicated to the probe, and written into non-volatile memory within the probe for future reference. Alternatively, it may be entered directed into the probe and communicated back to the main unit. The display also may show information relating to the probe's battery charge status, such as the amount of time the device has left of battery power. Such information may be relevant in some embodiments, for example, where an operator or user may be about to initiate an ultrasound exam and may need to change batteries before beginning the exam. The display also may provide low-battery warnings when the battery reaches a predetermined depleted state, for example. Also, the display may indicate any other operational errors with the system (i.e., main unit, probe, and/or other devices) during a diagnostic or self-test.

Instead of, or in addition to, providing a display indication related to the probe, some embodiments may have indications (e.g., LEDs) on the probe housing, main unit and/or other device. In some embodiments, it may not be desirable to have such indicators on the probe device because of the extra power drain on the battery that may result. In some embodiments, it may be desirable to provide detailed charge state information to the main unit at all levels of charge so the user can monitor and take appropriate action before the battery is depleted or nearly so. In these embodiments, by displaying a charge state on the main unit display instead of the probe device, there may be no additional battery discharge in the probe. Also, the display may permit a user to continuously or nearly so view the charge state during imaging, while still being able to view the remainder of the relevant information without interruption.

Power may be provided to the main unit, probe and other devices using a variety of techniques. For example, the main unit may operate on alternating current (AC) power, battery power or other alternative power sources. Similarly, the probe may operate on alternating current (AC) power, battery power or other alternative power sources. In the embodiments where the probe is wireless or thin-wire, or otherwise incapable of receiving power from an AC source for some period of time, it may be that the probe receives power from a battery, solar power, or other non-AC power source. Although the remainder of the disclosure may refer to battery power generally, it should be appreciated that such references include other power sources including, at least partially, AC power, solar power, and fuel cell sources. Because of the medically sensitive nature of the probe, it may be desirable to ensure that such battery power is available at all times. For example, a backup battery power source may be necessary in some embodiments.

In some embodiments, it may be desirable to conserve available probe power. Such conservation of energy may be limited to a certain period of time, in some embodiments. This may be accomplished using a variety of techniques contemplated by the disclosed embodiments. For example, the probe's circuitry may be turned off or powered down under certain predetermined conditions, like when such circuitry is deemed unnecessary, for example.

In some embodiments, the system may adapt to a change in battery charge by altering acquisition parameters and/or other system operating conditions. Such changing acquisition parameters may trade off image quality or frame rate for power usage, for example. For example, such changing acquisition parameters may include reducing the number of active receiver channels in the probe to reduce receiver power consumption. Reducing acquisition frame rate or transmit voltage may also lower power consumption, and hence, conserve battery power. Some embodiments may alert the user to changes in operating conditions caused by changes in battery charge state. For example, a message appearing on the system display may indicate a power saving mode level.

Similarly, in some embodiments, the system may adapt to the status of an optional thermal sensor located at the probe face by adjusting various system operating conditions to trade off image quality for lower transducer heat generation. Example parameters include the transducer drive voltage level, drive waveform, number of active transmitter elements, acquisition frame rate, etc.

In some embodiments, the main unit may operate on battery power, or perhaps also conserve electrical power usage. Therefore, like the probe, a main unit low-power state, a “standby” or “sleep” mode may be activated after some period of inactivity. The period of inactivity may be terminated automatically, by manual intervention, or some combination thereof. For example, in some embodiments a user may simply change the power status of the probe and/or main unit by pressing a button, or merely handling the probe via motion sensing (e.g., using an accelerometer and/or tilt switch, etc.). Also, the power status of the probe and/or main unit may be changed by the probe sensing a grip of the user's hand (e.g., by detecting heat, capacitance change, pressure, etc.). In some embodiments, it may be desirable to use a combination of sensing methods and/or to allow activation by deliberate operator action so it may not be triggered accidentally.

Other methods for conserving and controlling power status of the components in the system may include manual and/or automatic changing of power conditions (e.g., power off) to the components once a procedure is completed. The termination and/or changing of power conditions may be based on some predetermined period of time accrued by a timer in the system. For example, if a component like the probe is not operated for some period of time, the component may change its power state (e.g., turn itself off and/or place itself into a different power state). A different power state may include a relatively lower or higher power state. In some embodiments, this may be accomplished by changing the power state of a certain number of the portions of the probe or other device. For example, when imaging is in a “frozen” state (i.e., no live imaging) the probe's data acquisition and/or wireless interface transmitter circuitry may be turned off.

Initiating the change in power state may be accomplished in a number of ways all of which are contemplated by the disclosed embodiments. For example, some embodiments may contemplate various techniques for detecting a lack of activity, including the probe using motion, acceleration, heat and/or capacitance, or the like. Also, some embodiments may measure inactivity dictated by a period of time where controls on the probe, main unit, and/or other device are not operated. Also, following such inactivity, the component could power down either immediately and/or after some delay. The time period could be specified by the user and/or by some component in the system, including the probe, main unit or other device. Because in some embodiments, the main unit may communicate control information (e.g., periodically) to the probe, it may be desirable to allow the probe to detect a lack of control commands (e.g., over an extended period of time) from the main unit. For example, the probe may power itself down for a variety of reasons including because the main unit is either no longer turned on, is inoperable, and/or has been moved to a location out of wireless communication range, etc.

FIG. 10 provides just one example of a flow diagram 1000 for a probe inactivity timeout. As shown in FIG. 10, it is determined at 1001 whether an activation control has been activated. If, at 1001, it is determined that an activation control has not been activated, a loop will continue to check to see if an activation control has been activated. If, on the other hand, at 1001 an activation control has been activated, power is provided to the probe at 1002. At 1003, a timer is reset. The timer may be used to count to a predetermined time to determine if the probe has been inactive long enough to turn off the probe.

At 1004, it is determined whether a command is received by the probe, for example, from a main unit and/or another device. If, at 1004, a command is received by the probe, the timer is reset at 1003. If, on the other hand, at 1004, it is determined that a command is not received by the probe, it is determined at 1005 whether activation control is received by the probe. If, at 1005, it is determined that activation control is not received by the probe, the timer is reset at 1003. If, on the other hand, at 1005, it is determined that activation control is received by the probe, it is determined at 1005 whether a timeout has occurred. If, at 1005, it is determined at 1005 that a timeout has occurred, the probe is powered off at 1008. If, on the other hand, at 1005, it is determined at 1005 that a timeout has not occurred, at 1007, it is determined whether the timeout almost has occurred. If, at 1007, the timeout almost has occurred, the main unit may be informed of the impending timeout at 1009. If, on the other hand, at 1007, it is determined that the timeout almost has not occurred, the timer is reset at 1003.

In some embodiments, it may be desirable to permit the probe to remain active for some predefined time period after initial linkup, for example. It may be such that when the predefined period of time is about to run out, some indicator may be displayed for the user either on the probe (e.g., via a LED), the main unit (e.g., via a display), and/or both.

In addition, it should be appreciated that in some embodiments, the main unit and other devices may include similar non-AC power concerns and capabilities described above with regard to the probe.

In addition to providing and controlling power, some embodiments may include monitoring a charge or other status of the battery while in use and/or dormant. For example, in some embodiments, a controller may monitor the battery. The controller may be a separate part of the system and/or built into the battery pack. In some embodiments, the controller may track the characteristics of the battery and its use. For example, the controller may keep track of the amount of time the battery has been used, as well as the charge and discharge cycles. Also, the controller may provide feedback to the system and display such information to the user regarding the battery's current charge state. This may be accomplished, for example, by monitoring such parameters as the battery's open-circuit voltage, integrated current in and out since last full charge, etc. In some embodiments, such information may be transferred between the battery and probe or other devices using communication channels. Also, in some embodiments, estimating battery charge state may be accomplished using battery open-circuit voltage, load current integration over time (e.g., coulomb counting), and/or battery source resistance, for example.

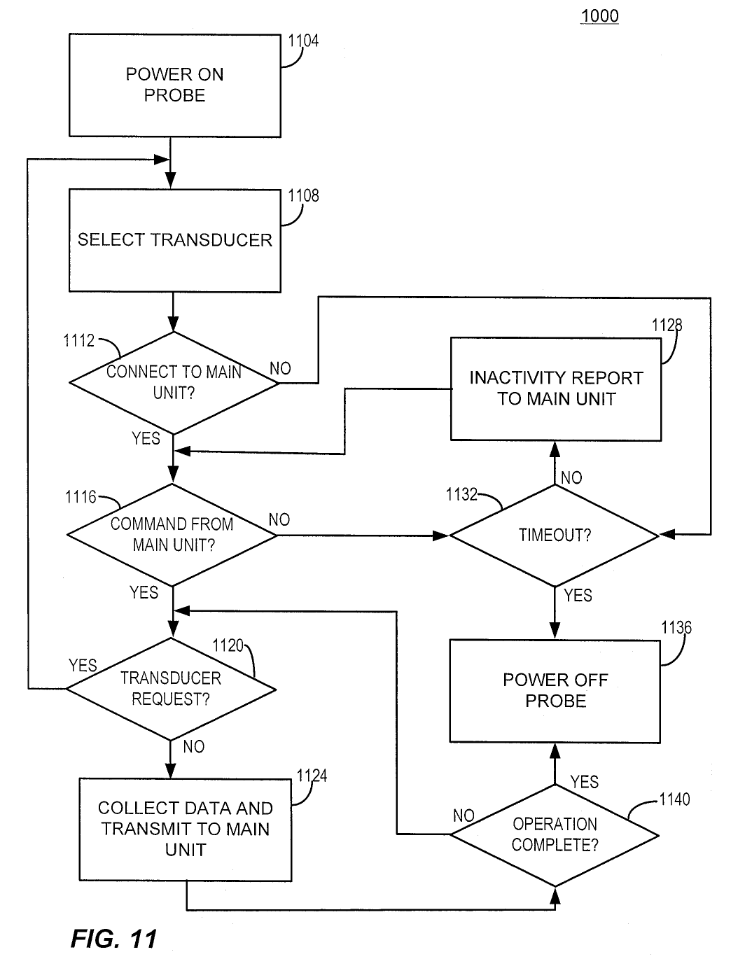

FIG. 11 illustrates an exemplary embodiment for the operational mode of a probe housing containing multiple transducer types. The probe housing switch is operable to supply power to the probe when place in the power on position 1004. The operator using the probe may, in a certain embodiment, select the transducer for use 1008 by placing the switch for the particular transducer type in the on position and placing the switch for any other type of transducer in the off position. The probe may then interrogate the main ultrasound unit to establish communication with the main unit 1012. If communication is not established the probe interrogates an internal timer to discover if the probe has been powered on longer than a pre-set time 1032 and if the power on with no activity pre-set time has been exceeded the probe may then power down 1036.

In this exemplary embodiment, if communication is successfully established with the main unit 1012, the probe may then check an incoming command queue for a operation command from the main ultrasound unit 1016. If there is no command in the queue for the probe to begin or continue operation, the probe may then be operative to interrogate an internal timer to discover is the probe has been powered on longer than the pre-set time for operation with no activity 1032. If the power on with no activity pre-set time has been exceeded the probe may then power down 1036 to conserve energy. If, however, in the exemplary embodiment a command to operate has been received from the main unit, the probe may be operative to check for a new transducer selection command 1020. If a new transducer select command has been received, probe operation returns to step 1008. If the transducer to be used is the currently selected transducer, the transducer begins to collect and process data for transmission to the main unit of the ultrasound system 1024. The transducer is operative to check for completion of the data collection 1040 either upon command from the main unit 1016. If data collection is complete, the probe is operative to power off 1036. If data collection is not yet completed, the probe may then check again for a request to change transducer types 1020 and continue operation 1024 until a completion signal is received from the main unit.

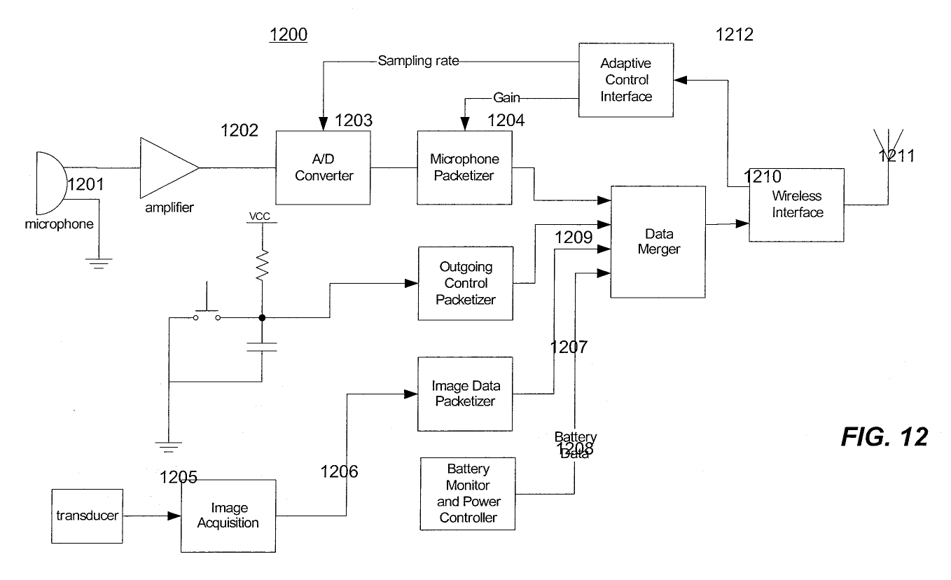

FIG. 12 is a block diagram illustrating data merger and adaptive control system 1200. As shown in FIG. 12, a microphone 1201 is in communication with an amplifier 1202. Amplifier 1202 is in communication with an analog-to-digital (A/D) converter 1203. A/D converter 1203 may operate to sample and digitize a signal from microphone 1201. A/D converter 1203 may have a sampling rate that may be adjusted by an adaptive control interface 1212 that may be responsive to a controller within the probe, the main unit, and/or another device. A microphone packetizer 1204 in communication with A/D converter 1203 may provide an adjustable number of bits per sample and/or dynamic range of the microphone signal data. Microphone packetizer 1204 also may encode the data in a compressed format using any number of standard and/or proprietary audio compression techniques (e.g., MP3) for further data reduction and possibly with variable compression parameters responsive to the adaptive control interface 1212. Microphone packetizer 1204 also may arrange the microphone audio data into discrete packets before merging with other data sources via a data merger 1210.

Also, as shown in FIG. 12, an outgoing control packetizer 1209 may receive control inputs from pushbuttons, knobs, trackballs, etc. and may arrange the associated control data into discrete packets before merging with other data sources via data merger 1210. Image data also may be packetized via an image data packetizer 1207 before data merger 1210. Image data packetizer may receive an image via transducer 1205 and image acquisition 1206. Battery status information may be generated by battery monitor and power controller 1208 function and passed to data merger 1210 to merge with other data sources. A thermal sensor (e.g., a thermistor) may be located where the probe makes contact with the body in order to sense probe temperature at the patient interface. The thermal sensing functionality may translate a signal from the thermal sensor into thermal status information to be sent to the main unit, for example, via data merger 1210. Both the battery and thermal status information may be made available in discrete data packets. Data merger 1210 may prioritize multiple data sources according to a predetermined and/or adaptively adjusted priority level. Data merger 1210 also may merge the data packets into one or more data streams leading to the wired and/or wireless interface 1211.

It should be appreciated that this description encompasses many types of probe designs including non-invasive, external probes as well as semi-invasive and/or invasive probes such as percutaneous, catheter-based, endo-cavitary, transesophageal, and/or laparoscopic probes in wired and/or wireless embodiments. For example, certain catheter-based, endo-cavitary, transesophageal, and/or laparoscopic probes are contemplated as wired and/or wireless probes.

Catheter-based ultrasound transducer probes may be used for intra-luminal and intra-cardiac ultrasonic imaging. There are various types contemplated by the disclosed embodiments including rotating single element, radial array and linear phased array. Rotating single element probes may be simpler to manufacture but may provide relatively poorer images due to their fixed focal depth. Also, in some embodiments, the scan plane rotating single element probes may be diametral to the catheter shaft. Linear arrays may be oriented along an axis of the shaft of the catheter, and therefore provide an image in a plane that is longitudinal to the catheter shaft. Linear arrays are typically more useful on larger vessels because they generally require a larger catheter shaft. Matrix and/or two-dimensional catheter-based transducers are also contemplated. In addition to side-fire methods, these may employ end-fire array geometries As is the case with other types of probes, in some embodiments, sterility may be desirable for catheter probes. As a result, embodiments that include a wireless catheter probe facilitate greater sterility by reducing and/or eliminating a need for a wired connection to the main unit.

In some embodiments, the probe or other components may be able to be configured, programmed and/or calibrated over the wired and/or wireless link from the main unit or other component. For example, in some embodiments, when the probe powers up for the first time, it may be that the wireless link and its support circuitry are fully functioning. The probe may include support circuitry that may be a field-programmable gate array (FPGA) with a boot EEPROM. The FPGA may be an Altera Cyclone™ FPGA or the like, that are provided with configuration data or calibration data. In some embodiments, the FPGA may be programmed from the wired or wireless interface without the need for a boot EEPROM. Alternatively, the boot EEPROM may be reprogrammable via the wireless interface to facilitate firmware updates. In this case, the FPGA may be initially programmed with the current EEPROM contents upon power up, after which time new programming code is loaded into the EEPROM from the wireless interface. The next time the probe is powered up, the FPGA may be loaded with the new EEPROM contents.

In some embodiments, other components like an acquisition controller, signal processing blocks and probe identification circuitry may be programmed after power up. After establishing the wireless link between the probe, main unit, and/or other components, an FPGA programming command may be communicated over the link to program the acquisition controller and the signal processing blocks. These blocks may also be reprogrammed to support different user input controls modes (i.e., color vs. b-mode, etc.) and/or reprogrammed to optimize for different tissue types and/or various other operating conditions. Reprogramming may occur while the image is frozen, on a frame-by-frame basis, and/or before each transmit event if necessary.

In some embodiments, a control interface for an FPGA may include control lines along with one or more data lines. Alternatively, if any of the hardware in the probe is a microcontroller, software could be downloaded in a manner similar to that described for the FPGA. Configuration tables for acquisition timing and coefficients for filtering and other signal processing functions may be loaded over the link. These configurations may be different for various user-controlled settings such as depth changes and/or mode changes, for example.

Configuration data or information may be provided from any of the components in the system Such configuration data, may include without limitation, power status, designation of device, type of device, frequency range, array configuration, power warnings, capability of a remote unit, quality of transmission of digital data, quantity of errors in transmission of digital data, availability of power required for transmission of digital data, change in transmission rate, completion of transmission, quality of data transmission, look-up tables, programming code for field programmable gate arrays and microcontrollers, transmission characteristics of the non-beamformed ultrasound wave, processing characteristics of the echoed ultrasound wave, processing characteristics of the digital data, and transmission characteristics of the digital data, etc.

Probe identification like serial number, probe type, bandwidth, revision number, and calibration data, for example, may be programmed into non-volatile memory either over the wireless link or a wired programming port. With respect to calibration, a calibration feedback loop may be initiated where acquired data is transmitted to the main unit to perform calculations. The main unit may then communicate such information as offset and gain data back to the probe where the data may be stored in a memory. In some embodiments, calibration may occur periodically and/or only once during probe production. In the latter case, the storage memory device may be non-volatile such as flash memory or EEPROM.

In some embodiments, it may be desirable to allow the user to locate a probe. For example, it may be that the probe is misplaced or the user needs to select one of many available probes and needs the proper probe to be distinguished for the operator. The system may include locator functionality that operates in a variety of ways contemplated by the disclosed embodiments. For example, some embodiments may include locator functionality with limited detection or geographic range, such that probes within the predetermined range (e.g., 10 meters) may be detected, while probes outside the range may be ignored in some embodiments. Also, the locators may have different characteristics, which may include active, passive or hybrid locator functionality, and the like.

Active locator functionality, for example, may include a receiver that may be of low power. The receiver would monitor (e.g., constantly, intermittently, etc.) for a particular electronic signature of the probe. For example, the electronic signatures may include RF emission characteristics, identification number, magnetic field signatures (e.g., magnetic field of circuitry, magnetic fields modulated with a particular signature, etc.).

In some embodiments, the probe may be identified to the user by a number of audible or visible techniques. For example, the system may emit an identifiable audible response, such as a beep for example, when it detects the proper probe (e.g., receives a particular RF signature). In some embodiments, the system may provide a visual indication, like the flashing of an indicator light when it detects the proper probe. Alternatively or in addition, it should be appreciated that these indicators may work by indicating an improper probe to prevent the user from selecting and using the wrong probe. It should also be appreciated that other techniques for providing indication of and locating for probes are contemplated by the disclosed embodiments. Also, in some embodiments, the indicators may be able to indicate a direction and/or distance that the user may travel to find the probe. For example, the indicator and locator functionality may use global positioning techniques, well known to those skilled in the art.

The communication between the locator components (e.g., receiver) may be the same wireless and/or wired channel used to communicate the image and/or control information between the probe, main system and other devices. Also, in some embodiments the locator functionality may have the option to use alternate communication channels.

Some embodiments may allow the locator communication channel to operate using techniques that allow reduced power consumption. For example, the locator's receiver may be powered for relatively shorter periods of time as needed, and then powered off when not needed (e.g., when waiting or after probe has been located).

Passive locator functionality also is contemplated by the disclosed embodiments. These passive techniques may not require active or powered circuitry in the probe, or other devices. This embodiment may be desirable where conservation of power in the system is a consideration. In this embodiment, for example, the locator functionality and components may produce an identifiable signature when placed into electrical and/or magnetic presence of an external source (e.g., an RF field).

In some embodiments, the external source may be attached to or housed in the main unit of the system and/or other systems or non-system devices. Also, the external source may be removable from being anchored to the system so as to facilitate searching for a lost probe. The external source may be AC powered or battery operated for greater portability. In some embodiments, the external source may emit a signal (e.g., a RF beacon signal). Some embodiments may use a signal having a particular frequency that is responsive with the passive receiver. As with the active locator functionality, upon detecting and/or locating the probe, an indication may be made to the user. Some embodiments may include the locator functionality within the probes such that one probe could be used to find another probe and/or to locate or distinguish itself, for example. For example, it may be able to ignore itself and find another probe by disabling the locator functionality while the probe is helping to find another probe.

It should also be appreciated that a combination of the passive and active techniques may be used in a hybrid system. For example, some embodiments may include a passive circuit sensitive to a particular RF signature that generates a trigger signal to activate a remainder of the locator components so that the probe can identify itself as described.

In some embodiments, the locator functionality may use relatively low-frequency RF, and magnetic coupling to communicate. In this way, the locator functionality may be able to operate over greater environmental circumstances and conditions. For example, using the low frequency, allows the generated magnetic fields to travel through more materials like conductive enclosures. In this way, the probe may be located even if it is placed in a metal cabinet, trash can and/or patient. Also, some embodiments may eliminate conditions like multi-path nulling by allowing the coupling between the antennae and devices to create a near-field phenomenon. In this way, the signal strength may be more accurately calculated as a function of distance and allow the locator functionality to be set to a power level that reliably covers a desired finding distance, yet not so far as to stimulate probes at a greater distance.

In some embodiments, because of the relatively lower frequency, the required power of the locator circuit may be reduced. For example, the power level may be nominal as compared to battery capacity. In this way, in some embodiments, the locator functionality may be run continuously (or nearly so) as may be necessary to find a lost probe, yet use relatively little battery power.

While the embodiments have been described in connection with various embodiments of the various figures, it is to be understood that other similar embodiments may be used or modifications and additions may be made to the described embodiment for performing the same function of the disclosed embodiments without deviating therefrom. Therefore, the disclosed embodiments should not be limited to any single embodiment, but rather should be construed in breadth and scope in accordance with the appended claims.

What is claimed is:

1. A probe, comprising:
a first transducer having a first characteristic, wherein the first transducer transmits a first pressure wave;
a second transducer having a second characteristic, wherein the second transducer transmits a second pressure wave;
a housing substantially incorporating the first transducer and the second transducer, wherein the first transducer is located at an opposite end of the housing with respect to the second transducer.

2. The probe of claim 1, wherein the first characteristic is different than the second characteristic.

3. The probe of claim 1, further comprising a transceiver that receives an echoed ultrasound wave and converts the received echoed ultrasound wave into digital data, and wherein the transceiver communicates the digital data.

4. The probe of claim 3, wherein the transceiver wirelessly communicates the digital data.

5. The probe of claim 3, wherein the transceiver communicates the digital data over a wire.

6. The probe of claim 5, wherein the wire includes at least one of the following: electrical conductor, magnetic conductor, twisted pair of conductors, coaxial conductor, or optical conductor.

7. The probe of claim 3, further comprising a multiplexer in communication with the transceiver.

8. The probe of claim 7, wherein the multiplexer arranges the digital data by manipulating the digital data after converting the received echoed ultrasound waves to digital data.

9. The probe of claim 7, wherein the multiplexer arranges the digital data via interleaving.

10. The probe of claim 7, wherein the multiplexer uses at least one of the following techniques: time-division multiplexing, frequency-division multiplexing, code-division multiplexing, or pulse width-division multiplexing.

11. The probe of claim 3, further comprising storing the digital data at a rate that varies as a function of at least one of the following: an available bandwidth of the transmission, a rate at which the digital data is acquired, an image frame, an interruption of transmitting the digital data, a transmit aperture, or a receive aperture.

12. The probe of claim 1, further comprising a battery.

13. The probe of claim 1, wherein the first and second characteristics each comprise at least one of the following: dimensions, configuration, center frequency, frequency range, element pitch, element size or element type.

14. The probe of claim 1, wherein the transducers are configured as an array, and wherein the array is at least one of the following: curved or straight.

15. The probe of claim 1, further comprising an indicator unit that provides indication of at least one of the following: power status, designation of unit, type of unit, frequency range, array configuration, power warnings, capability of unit, quality of transmission of digital data, quantity of errors in transmission of digital data, availability of power required for transmission of digital data, change in transmission rate, completion of transmission, quality of data transmission, processing characteristics of the digital data, or transmission characteristics of the digital data.

16. The probe of claim 1, wherein the second transducer is at least one of the following: a sound sensor or a receiver.

17. The probe of claim 3, further comprising adjusting a displayed frame rate based on a rate of transmission of the digital data.

18. The probe of claim 3, further comprising adjusting an amount of digital data per image frame based on a rate of transmission of the digital data.

19. The probe of claim 3, further comprising reducing a bandwidth of the digital data by at least one of the following: converting a received echo ultrasound wave to a different frequency band prior to converting to digital data, quadrature sampling, second order sampling, or third order sampling.

20. The probe of claim 3, further comprising packetizing the digital data.

21. The probe of claim 3, further comprising transmitting the digital data using at least one of the following techniques: optical, infrared, radio frequency, and ultrawideband frequency.

22. The probe of claim 1, further comprising a sensor unit for sensing a proximity of the housing to a remote unit using at least one of the following techniques: optical, infrared, capacitive, inductive, electrically conductive, or radio frequency.

23. The probe of claim 22, further comprising providing power to the first transducer and the second transducer upon sensing a proximity to the housing.

24. The probe of claim 3, further comprising transmitting a unique identifier with the digital data, wherein the unique identifier is used for at least one of the following: initiating communication with a remote unit, synchronizing communication with a remote unit, or ensuring communication with a predetermined remote unit.

25. The probe of claim 1, wherein first transducer and second transducer are separated by a predetermined distance within the single housing.

26. The probe of claim 1, wherein the first transducer and the second transducer operate in an ultrasound range.

27. A method for conducting ultrasound interrogation, comprising:
transmitting a first pressure wave from a first location; and
transmitting a second pressure wave from a second location,
wherein the first location and the second location are substantially within a single housing, and wherein the first location is at an opposite end of the housing with respect to the second location.

28. The method of claim 27, further comprising providing power to the first transducer and the second transducer upon sensing a proximity to the housing.

29. The method of claim 27, wherein the first and second characteristics each comprise at least one of the following: dimensions, configuration, center frequency, frequency range, element pitch, element size or element type.

30. The method of claim 27, wherein the second transducer is at least one of the following: a sound sensor or a receiver.

31. The method of claim 27, wherein the first transducer has a first characteristic, and wherein the second transducer has a second characteristic, and wherein the first characteristic is different than the second characteristic.

32. The method of claim 27, further comprising receiving an echoed ultrasound wave, converting the received echoed ultrasound wave into digital data.

33. The method of claim 32, further comprising wirelessly communicating the digital data.

34. The method of claim 32, further comprising communicating the digital data over a wire.

35. The method of claim 34, wherein the wire includes at least one of the following: electrical conductor, magnetic conductor, twisted pair of conductors, coaxial conductor, or optical conductor.

36. The method of claim 32, further comprising multiplexing the digital data.

37. The method of claim 36, further comprising arranging the digital data by manipulating the digital data after converting the received echoed ultrasound waves to digital data.

38. The method of claim 36, further comprising multiplexing the digital data via interleaving.

39. The method of claim 36, further comprising multiplexing the digital data using at least one of the following techniques: time-division multiplexing, frequency-division multiplexing, code-division multiplexing, or pulse width-division multiplexing.

40. The method of claim 32, further comprising storing the digital data at a rate that varies as a function of at least one of the following: an available bandwidth of the transmission, a rate at which the digital data is acquired, an image frame, an interruption of transmitting the digital data, a transmit aperture, or a receive aperture.

41. The method of claim 27, further comprising providing an indication of at least one of the following: power status, designation of unit, type of unit, frequency range, array configuration, power warnings, capability of unit, quality of transmission of digital data, quantity of errors in transmission of digital data, availability of power required for transmission of digital data, change in transmission rate, completion of transmission, quality of data transmission, processing characteristics of the digital data, or transmission characteristics of the digital data.

42. The method of claim 32, further comprising adjusting a displayed frame rate based on a rate of transmission of the digital data.

43. The method of claim 32, further comprising adjusting an amount of digital data per image frame based on a rate of transmission of the digital data.

44. The method of claim 32, further comprising reducing a bandwidth of the digital data by at least one of the following: converting a received echo ultrasound wave to a different frequency band prior to converting to digital data, quadrature sampling, second order sampling, or third order sampling.

45. The method of claim 32, further comprising packetizing the digital data.

46. The method of claim 32, further comprising transmitting the digital data using at least one of the following techniques: optical, infrared, radio frequency, and ultrawideband frequency.

47. The method of claim 27, further comprising sensing a proximity of the housing to a remote unit using at least one of the following techniques: optical, infrared, capacitive, inductive, electrically conductive, or radio frequency.

48. The method of claim 32, further comprising transmitting a unique identifier with the digital data, wherein the unique identifier is used for at least one of the following: initiating communication with a remote unit, synchronizing communication with a remote unit, or ensuring communication with a predetermined remote unit.

49. An ultrasound probe, comprising:
a first transducer array having a first characteristic, wherein the first transducer array transmits a first ultrasound wave;
a second transducer array having a second characteristic, wherein the second transducer array transmits a second ultrasound wave;
a transceiver in communication with the first transducer array and the second transducer array, wherein the transceiver receives an echoed ultrasound wave and converts the received echoed ultrasound wave into digital data, and wherein the transceiver communicates the digital data; and
a housing substantially incorporating the first transducer and the second transducer.

50. The ultrasound probe of claim 49, wherein the transceiver communicates the digital data via at least one of the following: wirelessly or over a wire.

51. The ultrasound probe of claim 49, wherein the first transducer array is located at an opposite end of the housing with respect to the second transducer array.

52. The ultrasound probe of claim 49, wherein the first transducer array is located at an adjacent end of the housing with respect to the second transducer array.

53. The ultrasound probe of claim 49, wherein the first transducer array is located parallel with respect to the second transducer array within the single housing.

54. The ultrasound probe of claim 49, wherein the first characteristic is different than the second characteristic.

55. The ultrasound probe of claim 49, wherein the first transducer array is configured to be used in a different medical application than the second transducer array.

56. The ultrasound probe of claim 49, further comprising a multiplexer in communication with the transceiver.

57. The ultrasound probe of claim 56, wherein the multiplexer arranges the digital data by manipulating the digital data after converting the received echoed ultrasound waves to digital data.

58. The ultrasound probe of claim 56, wherein the multiplexer arranges the digital data via interleaving.

59. The ultrasound probe of claim 56, wherein the multiplexer uses at least one of the following techniques: time-division multiplexing, frequency-division multiplexing, code-division multiplexing, or pulse width-division multiplexing.

60. The ultrasound probe of claim 49, further comprising storing the digital data at a rate that varies as a function of at least one of the following: an available bandwidth of the transmission, a rate at which the digital data is acquired, an image frame, an interruption of transmitting the digital data, a transmit aperture, or a receive aperture.

61. The ultrasound probe of claim 49, further comprising adjusting a displayed frame rate based on a rate of transmission of the digital data.

62. The ultrasound probe of claim 49, further comprising adjusting an amount of digital data per image frame based on a rate of transmission of the digital data.

63. The ultrasound probe of claim 49, further comprising reducing a bandwidth of the digital data by at least one of the following: converting a received echo ultrasound wave to a different frequency band prior to converting to digital data, quadrature sampling, second order sampling, or third order sampling.

64. The ultrasound probe of claim 49, further comprising a sensor unit for sensing a proximity of the housing to a remote unit using at least one of the following techniques: optical, infrared, capacitive, inductive, electrically conductive, or radio frequency.

65. The ultrasound probe of claim 64, further comprising providing power to the first transducer and the second transducer upon sensing a proximity to the housing.
